(19) United States
(12) Patent Application Publication
(10) Pub. No.: US 2021/0353958 A1
(43) Pub. Date: Nov. 18, 2021

(54) DEVICE FOR TREATING PATIENTS BY PHOTOTHERAPY

(71) Applicants: MDB TEXINOV, Saint Didier De La Tour (FR); DELTAVAL, Lyon (FR)

(72) Inventors: Sandra Berger, Fitilieu (FR); Charlotte Donge, Saint Jean De Soudain (FR); Anne Plat, Dagneux (FR); Jacques Tankere, Meximieux (FR)

(21) Appl. No.: 17/287,433

(22) PCT Filed: Oct. 25, 2019
(86) PCT No.: PCT/FR2019/052549

§ 371 Date: Apr. 21, 2021

(30) Foreign Application Priority Data
Oct. 31, 2018 (FR) 1860085

(51) Int. Cl. A61N 5/06

(52) U.S. Cl. CPC A61N 5/0621; A61N 2005/063; A61N 2005/0663; A61N 2005/0645; A61N 2005/0637

(57) ABSTRACT

A device for the treatment of patients by phototherapy includes a first multiple-use portion, formed of a flexible textile structure, including radiation generators formed by knitting and integrating optical fibers. At least one end of the optical fibers is connected to a light source, and the optical fibers are integrated in the knit stitches or positioned in partial wefts in the knit, to emit light due to the exceeding of their limiting bending angle, according to the so-called “macro-bending” technology. A second single-use portion, removably associated with the first portion, is intended to come into contact with the treated patient, and capable of diffusing towards the latter the light emitted by said first portion.

### TECHNICAL FIELD

The invention concerns the field of the treatment of patients by phototherapy. More particularly, the invention aims at the treatment of the jaundice of newborns or prematurely-born children.

It particularly aims at a phototherapy treatment which is at the same time simple to implement, confining the light emitted by such a device to the areas of the patient's body meant to be submitted to the treatment, and further ensuring an optimized security, particularly when the patient is a newborn or an infant.

### BACKGROUND

Neonatal icterus, appearing as a yellow coloring of integuments due to the accumulation of bilirubin in the organism, also better known as jaundice, is relatively common.

Different treatments are known to heal this disease, and particularly:

for a light jaundice, the treatment comprises having the newborn suckle abundantly to favor the elimination of bilirubin via the feces and urines;

drug therapies by the implementation of enzymatic inducers, and perfusion of albumin to capture the bilirubin;

exchange transfusion, that is, the replacement of a significant portion of the newborn's blood;

phototherapy, that is, submitting the newborn to a non-ionizing radiation, having a wavelength typically in the range from 420 to 490 nanometers, said light degrading the bilirubin.

In the field of phototherapy, one of the techniques conventionally used comprises positioning the newborn in a tunnel, under lamps emitting blue light to ensure such a phototherapy treatment, the infant's eyes being masked by any appropriate means, and the genitals being protected. The time of exposure of the infant in this tunnel may vary by several hours in a row, and this for several days according to the degree of severity of the jaundice.

This technique has a number of disadvantages, among which the following can be mentioned:

the separation for several hours in a day of the mother and of the child, affecting the essential link developing between them from as soon as after birth;

the need to hospitalize the child in a pediatric department and not in a maternity department if the jaundice is only detected after coming out of the maternity department;

the heat generation inherent to lamps or radiating systems located inside of such tunnels, likely to cause burns, in particular when halogen lamps are implemented, and in extreme cases hyperthermia or even the newborn's death, requiring associating with these devices temperature alarm and heat dissipation ventilation systems;

a non-uniform exposure of the newborn's body, inherent to the specific shape of these tunnels, particularly convex;

the necessary interruption of the duration of exposure of the newborn, due to the indispensable nursing of the child, particularly in terms of feeding.

Due to these different disadvantages, another technique comprising implementing flexible textile devices, enabling to first drastically decrease manufacturing costs, and to further significantly optimize the comfort, particularly for the infant, has been provided.

Document KR 2016 01 431 81 for example describes a device appearing in the form of a blanket, having an array of LED diodes coupled by metal wires integrated therein. However, due to its manufacturing mode, this device remains relatively stiff and looses the conformability which is desired to be obtained by the implementation of a flexible technology. Moreover, the array of LED diodes is not continuous, so that the homogeneity of the light at the level of the infant's body is not satisfactory. Finally, the presence of such LED diodes directly within the cover generates a heating risk, requiring the integration of temperature sensors and, during its implementation, the monitoring of the infant.

To optimize the conformability of such a phototherapy device, it has been provided, for example, in document CN 106861048, to implement a phototherapy device integrating a textile structure emitting light obtained by weaving of laterally-emitting optical fibers.

It can however be observed that this textile is relatively complex to form and requires, to obtain the desired illumination homogeneity, implementing optical fibers filled with titanium dioxide nanofillers, having a microcrystal nanocoating added thereon by corona treatment. These different treatment steps make such a device expensive, thus making it rather unaffordable for nursing centers. Further, the embodiment of such a device rigidifies the light-emitting textile, which is antinomical with the desired results.

A light-diffusing multilayer flexible envelope, formed on a textile base woven with laterally-emitting optical fibers, has also been provided in document WO 2013/068518. If, undoubtedly, this device has an improved flexibility, and thus enables to optimize the newborn's comfort, it however requires including phase-change microcapsules intended to regulate the temperature, increasing manufacturing costs. Further, the device described in this document does not enable to do away with risks for the newborn to be buried into the envelope, with the accompanying suffocation risks.

Document WO 2016/012732 describes a flexible textile device formed by weaving with laterally-emitting optical fibers. Although here again, this device is an improvement in terms of flexibility and accordingly of comfort for the infant, the problem of the management of the heating inherent to light diffusion remains. It is indeed advocated, to fight this heating, to form a volume defined by the arranging of lateral walls with openings to ensure an air flow, but which accordingly generates light leaks, and which here again requires a significant attention regarding the risk of hyperthermia.

As a summary, there does not exist to date a flexible phototherapy treatment device combining, in particular, the comfort and well-being of the patient, and in the case in point of the infant, with the efficiency of the treatment in optimized security conditions.

### SUMMARY OF THE DISCLOSURE

The invention endeavors to respond to these different requirements.

It concerns a flexible-type device for the treatment of patients by phototherapy.

It aims at responding to the following requirements:

adjustment of the quantity of emitted light;

homogeneity of the emitted light all over the patient's body;

limitation, or even elimination, of the heat generated by the structure;

optimization of the patient's comfort;

optimization of the mother-child contact;

integration of a system for preventing the newborn's burying within the device;

limitation, or even elimination, of any light emitted towards the outside of the device;

possibility of very easily extracting the newborn, particularly effortless and in a single gesture and very rapidly in case of medical distress;

a system guaranteeing a high level of hygiene to respond to risks of nosocomial diseases which develop in hospitals.

For this purpose, the invention provides a device for the treatment of patients by phototherapy comprising:

a first multiple-use portion, formed of a flexible textile structure, comprising radiation generators formed by knitting and integrating optical fibers, at least one end thereof being connected to a light source, said optical fibers being integrated into the knit stitches, or positioned in partial wefts in the knit to emit light in the wavelength of interest due to the exceeding of their limiting bending angle, according to the so-called “macro-bending” technology;

a second single-use portion, removably associated with said first portion, and intended to come into contact with the treated patient, and capable of diffusing towards the latter the light emitted by said first portion.

In other words, the invention first comprises providing two distinct flexible structures, reversibly associated with each other:

one intended to generate the light radiation in the desired wavelength range, and thus integrating at least one radiation generator, having a structure such that it enables to obtain a homogeneity of the illumination of the patient over a significant portion of their body;

and the other intended to be single-use, and thus typically formed of a disposable cover, this, to keep intact said radiation generation device, easily separable therefrom, and thus to favor the use of said device in hygiene conditions acceptable in the considered medical field.

According to the invention, the structure of the first multiple-use portion defines a light surface enabling to illuminate at least 60% of the surface area of the patient's body.

According to another aspect of the invention, the optical fibers implemented within said first multiple-use portion, that is, within the structure generating the radiation, are knit so that said optical fibers are bend beyond the limiting bending angle of said fibers. This limiting bending angle intrinsically depends on the material forming the optical fiber, as well as on the diameter thereof. Thus, for a considered optical fiber, the light emission is performed in the bending plane and is dependent on the stress applied to said fiber. In other words, and conversely to prior art devices, due to the absence of direct light outlet, there is no heating, accordingly significantly optimizing the security of the device and simplifying its use since it is no longer necessary to implement the monitoring of the infant, and generally of the patient, during the treatment phase. To have a constant diffusion intensity with this technology, which is besides desired in the context of the present invention, the limiting bending angle of said optical fibers, should be able to be kept during the implementation of the textile integrating them.

This technology has been described, particularly in WO 2016/097524. In the case in point, as already mentioned, the optical fibers are bent beyond their limiting bending angle, the knit into which they are integrated being obtained by warp knitting or Rachel or crocheting technology. The number of weaves of the knit enables to maintain the desired bending of the optical fibers, while providing a given flexibility and a given extensibility of the structure, and accordingly enables to control the quantity and the quality of emitted light. Further, this technology enables to implement optical fibers of still smaller diameter, accordingly giving the textile into which they are integrated an increased flexibility, and to the device a higher conformation degree.

According to the invention, the first multiple-use portion integrating the radiation generators, comprises a dorsal wall and a ventral wall, coupled to each other at the level of their respective lower portion, thus defining a hinge.

Advantageously, each of the dorsal and ventral walls comprises at the level of their upper end a cutout capable of defining a neck-line type opening, intended to enable the patient's head to emerge out of the device while limiting leaks of the light emitted towards the patient's head.

According to the invention, the second single-use portion is formed of a double pocket, having dimensions substantially equal to or slightly greater than those of the dorsal and ventral portions of said first multiple-use portion, each of the two pockets being intended to slip over said ventral and dorsal portions, whereby the above-mentioned term cover; the inner surface of this second single-use portion, intended to come into contact with the patient, is continuous; in other words, the two pockets are attached to each other by said continuous inner surface.

Further and according to the invention, the inner surface of the second single-use portion, intended to come into contact with the patient's back, is provided with a system for preventing the newborn's burying, that is, with means capable of opposing the sinking down of said newborn in the device of the invention, the obvious risk thereof being for the infant to suffocate. This system is formed of a band intended to pass between the infant's legs, the free end thereof being reversibly attached to said newborn, typically to the diaper that the latter is generally provided with. This results, in addition to the desired burying prevention function, in a great simplicity of positioning of the infant within the device of the invention whatever their size, and accordingly in a great ease of extraction of the newborn out of the device. Typically, this reversible attachment mode may be formed of a hook and loop system, better known under trademark VELCRO.

Advantageously, the free ends of one of the two pockets forming the second single-use portion are provided with flap-type reversible attachment means, capable of cooperating with the other pocket and intended to limit leaks of the light emitted by the device towards the patient's head.

It will have been understood that the invention also aims at limiting, or even suppressing, any leaks of light out of the volume intended to receive the patient and, in the case in point, the infant.

### BRIEF DESCRIPTION OF THE DRAWINGS

The foregoing features and advantages of the present invention will now be discussed in the following non-limiting description of a specific embodiment, in relation with the accompanying drawings.

FIG. 1 is a simplified perspective representation of the device of the invention.

FIG. 2 is a simplified representation of the first multiple-use portion of the device of the invention in open position.

FIG. 3 is a simplified representation in partial cross-section of the device of the invention in semi-open position.

FIG. 4 schematically illustrates the device of the invention with the burying prevention means.

### DETAILED DESCRIPTION

The flexible phototherapy treatment device of the invention has thus been schematically shown in the drawings. Basically, it appears, in a way, in the form of a sleeping bag or jumpsuit, since it is mainly intended to be used for newborns. It is formed of two flexible structures, reversibly associated with each other:

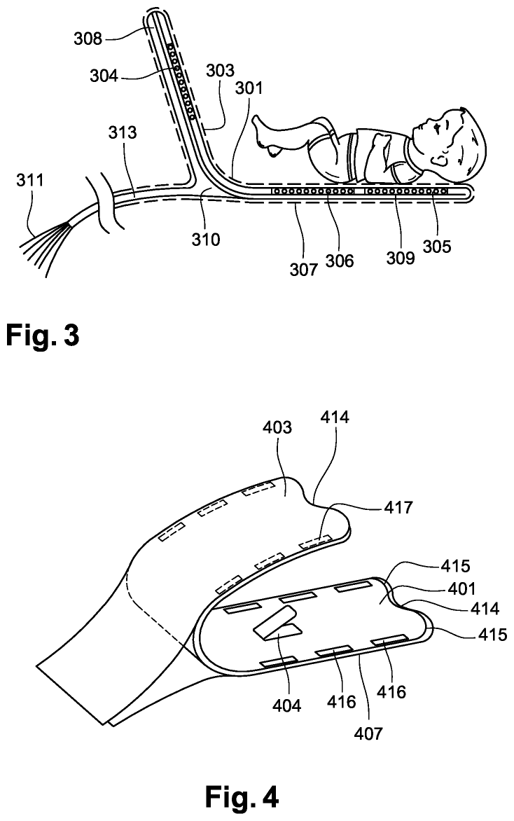

a first so-called multiple-use structure (200), and in practice integrating three light or radiation generators (204, 205, 206; 304, 305, 306), described in further detail in relation with FIGS. 2 and 3;

and a second “disposable”, and thus single-use, structure (103; 303, 307; 403, 407), particularly meant for hygiene purposes on the one hand, and for the patient's comfort on the other hand. Typically, it is a cover, since it is meant to protect said first structure, particularly against the newborn's feces and urines.

The light generator is formed of at least two knit textile structures (204, 205, 206; 304, 305, 306), sewn on a membrane (208, 209; 308, 309), defining a dorsal wall (209, 205, 206; 309, 305, 306) and a ventral wall (208, 204; 308, 304) coupled to each other at the level of their respective lower end and thus forming a hinge (210, 310).

The light generator is, as already mentioned, formed of a knit implementing the Rachel warp or crocheting knit technology integrating optical fibers, according to a weave such that their bending exceeds their limiting bending angle, to emit said light in the bending plane. For this purpose, the optical fibers are either totally integrated into the knit stitch to obtain an intense point-like light, or positioned in a partial weft with a constant pitch, to obtain a homogeneous light having a substantially uniform intensity across the entire surface of said textile.

The knit integrating the optical fibers is typically made of polyester. However, any other material may be implemented, such as linen, cotton, polyamide, etc.

The membrane (208, 209; 308, 309) having these light generators sewn thereon is for example a bilayer comprising:

an inner layer formed of a thin, while, shiny or matt, impermeable and breathable membrane, first intended to direct the light generated by the light generator towards the patient, and to prevent leaks of light towards the outside of the device, such as for example, a waterproof-breathable membrane having a typical thickness in the range from 50 to 400 micrometers; this membrane may for example be made of polyurethane;

and an outer layer, advantageously dark-colored, to block the little light likely to diffuse through the inner layer, for example, formed of a polyester textile having a density in the range from 75 to 250 g/m².

The inner layer advantageously complies with the Oekotex 100 class 1 or the like standard https://www.oeko-tex.com/fr/business/certifications_and_services/ots_100/ots_100_product_classes/ots_100_product_classes.html; Product class textiles and textile toys for babies and children up to and including 3 years old, for example, undergarments, jumpsuits, sheet, bedding products, fabric animals, etc.

The dorsal and ventral walls of this membrane are attached to each other by sewing at the level of a hinge area (210, 310).

The optical fibers integrated in the knit textile structures forming the radiation generators are gathered in a bundle (111, 211, 311) of optical fibers, each of said optical fibers being connected (112, 212) to a LED-, OLED-, or laser-type light source (not shown), having a wavelength corresponding to the latest spectrum recommendations for the treatment of jaundice; currently, this spectrum is typically in the range from 420 to 500 nanometers, but the invention is capable of adapting to other medical recommendations.

As can be seen in FIGS. 2 and 3, and to guarantee the absence of any contact between the free optical fibers, that is, those which are not integrated in the radiation generators, and the newborn, the bundle of said optical fibers is surrounded with a sleeve (113, 213, 313) which ends at the level of the connection or hinge area (210, 310) between the dorsal portions and of the ventral portion defining said first multiple-use structure.

The free upper ends of the walls, respectively dorsal and ventral, of said first multiple-use structure exhibit a cutout (214) defining, when they are reversibly attached to each other via said second single-use structure (see hereafter) a neck line from which the infant's head is likely to emerge. For this purpose, the single-use structure has equivalent cutouts (114, 414) provided with a hook and loop fastening system (of Velcro® type), in order to, on the one hand, prevent the infant's head from penetrating into the space defined by said dorsal and ventral ails, avoiding the risks for the infant to bury within this space, and on the other hand to prevent the light generated in said space from coming out thereof, and accordingly from illuminating the newborn's head, and thus particularly their eyes

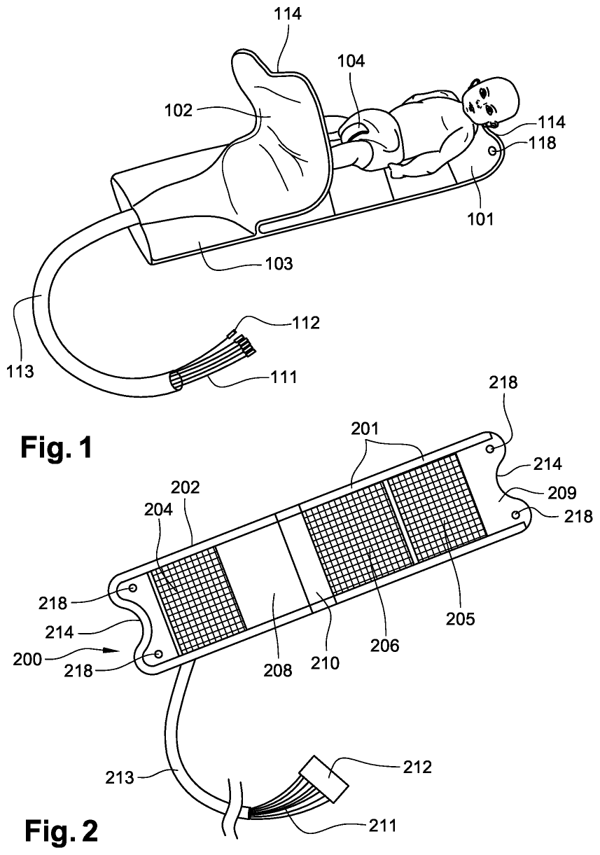

According to the invention, this first structure or multiple-use portion is intended to removably receive a second single-use structure, typically disposable. The latter defines two pockets (303, 307) intended to slip on at the level of the two dorsal and ventral walls of said first multiple-use portion, and thus of dimensions substantially corresponding, at least widthwise and to within the clearance, to the dorsal and ventral walls of the multiple-use structure to ease the slipping into said pockets. However, and as can be well observed in FIGS. 1, 3, and 4, the inner surface (101, 301, 401) of this disposable portion is continuous, to preserve the multiple-use portion from any soiling (urine, feces) A dorsal pocket (307) and a ventral pocket (303) are thus defined. Due to the implementation of this second single-use disposable portion, hygiene conditions are optimized and problems of cleaning and of risk of contamination of the essential structure of the device, that is, of the light generator, are avoided.

This disposable portion or cover is typically made of a multi-material, typically integrating a non-woven and a waterproof-breathable layer generally having light passage properties of from 80 to 100% in its entirety, enabling the light emitted by the light generators to diffuse through towards the infant.

This multilayer is typically a bilayer, respectively comprising:

a surface intended to come into contact with the patient's skin, and thus soft and absorbing, for example of non-woven type or of an absorbing and non-irritating material for example made of PET (polyethylene terephthalate) microfibers or of natural material; and

a surface in contact with the light generators, impermeable to protect the latter from possible soiling (urines, feces) and preserve the hygiene conditions of the device of the invention, for example, a transparent waterproof-breathable membrane; this surface is for example formed of a polyurethane membrane.

These two surfaces may be laminated together by thermobonding or by a glue array. The assembly thus formed allows a good light transmission in the order of 85 to 98% towards the infant.

This disposable portion comprises burying prevention means formed of a band (104, 404) originating from the dorsal portion and capable reversibly attaching, typically to the infant's diaper, by means of a hook and loop system. The great ease of positioning of the infant on their back on the dorsal portion of said first multiple-use portion coated with the disposable cover, and then of positioning of this band on the infant's diaper at the desired height to allow their head to emerge out of the device of the invention can thus be imagined.

Further, the free ends of said disposable portion comprise a cutout intended to define the neck line, similarly to the cutout made at the upper end of the dorsal and ventral walls of the multiple-use structure and for the same purpose, that is, the emergence of the infant's head out of the device of invention (see above).

Further, the free ends of the dorsal pocket of the disposable portion comprise a flap (415) provided on its inner surface with a reversible hook-and-loop type attachment system, intended to cooperate with the free ends of the outer surface of the ventral pocket, its non-woven material allowing the adherence with the hook and loop system. Any coming out of light is thus avoided at the level of the neck line thus defined, enabling to avoid for the infant to wear glasses or a light-protection band. In other words, the light is confined within the device of the invention when the latter is in place around the infant.

This design of the disposable or single-use portion thus provides a very high guarantee against the risk of burying of the newborn, who thus cannot sink down in the device due to a double security resulting, on the one hand, from the support band passing between their legs and attached to the diaper by the hook and loop system, and on the other hand, by the closing of the flaps formed at the upper end of the single-use structure, which, once positioned, do not allow the passage of the infant's head into the device.

The single-use disposable portion further comprises, either on its dorsal portion, or on its ventral portion, a reversible hook-and-loop-type attachment system (416, 417) appearing in the form of bands formed along the two lateral edges of the considered portion, and intended to cooperate with the non-woven surface of the other portion. Thereby, the confinement of light inside of the device of the invention is ensured in cooperation with the previously-described neck line system. Further, due to this reversible attachment mode, the extraction of the infant out of said device in case of emergency is made easy.

Further, the second single-use portion may advantageously comprise an emitter of RFID, radio tag, or the like type, capable of being activated by the light source then integrating, according to a well known technology, a transceiver adapted to the frequency of the tag.

This technical solution enables to control the presence or the systematic changing of the single-use portion, guaranteeing, on the one hand, a security of use, and, on the other hand, the general hygiene of the device for the patient.

The automation already integrated in the device guarantees the lack of activation of the light source in the absence of the disposable portion or when said disposable portion has already been used, to guarantee the hygiene by its presence and to avoid any risk of contamination between infants.

As a variant, all the hook and loop systems described hereabove may be replaced with other fastening means of snap fastener type or any other fastening system fast and simple to release in a single move, to allow care in case of a vital emergency for the infant.

Advantageously, the disposable portion is reversibly attached to the multiple-use portion by means of snap fasteners (118, 218) positioned at the respective ends of the dorsal and ventral portions of the disposable portion and of the multiple-use portion.

The advantage of the phototherapy treatment device according to the invention can be understood.

First, it complies with all medical requirements in terms of:

irradiance: treatment efficiency;

safety of the newborn: no generated heat, presence of a burying prevention system without for all this hindering the ease of extraction of the infant from the device with a simple and fast gesture in case of an emergency;

absence of light leaks, thus avoiding the discomfort of wearing glasses or a band on the eyes;

hygiene due to the presence of the second single-use portion, typically forming a disposable cover;

double security system concerning the burying prevention and the ultra-fast release of the infant from the device, to allow care in case of a vital emergency, whatever its cause.

The device of the invention further enables to significantly optimize the infant's comfort and well-being:

indeed, although it is coupled to a light source, the device enables to carry the child, and thus to ensure a parent-to-child contact;

it can be envisaged to use this device in a home treatment, to thus avoid hospitalizations in pediatric departments and separating the mother from the child since it is then no longer necessary to monitor the child during treatment phases.

the treatment time may be optimized with respect to the implementation of a conventional tunnel since it becomes for example possible to feed a child during the treatment.

1. A device for the treatment of patients by phototherapy, comprising:
a first multiple-use portion formed of a flexible textile structure, comprising radiation generators formed by knitting and integrating optical fibers, at least one end thereof being connected to a light source, said optical fibers being integrated in knit stitches, or positioned in partial wefts in the knit, to emit light due to the exceeding of their limiting bending angle, according to the so-called “macro-bending” technology;
a second single-use portion removably associated with said first portion, configured to come into contact with the treated patient, and capable of diffusing towards said patient the light emitted by said first portion.

2. A device for the treatment of patients by phototherapy according to claim 1, wherein the first multiple-use portion comprises a dorsal wall and a ventral wall, each wall being provided with a lower portion, said respective lower portions forming an hinge connecting said walls to each other and each wall comprising at the level of their upper end a cutout capable of defining a neck-line type opening, configured to enable a patient's head to emerge out of said device, while limiting leaks of the light emitted by said first portion towards the head, on which dorsal and ventral walls the light generators are arranged.

3. A device for the treatment of patients by phototherapy according to claim 2, wherein the second single-use portion is formed of a double pocket, each of the two pockets being configured to slip on the ventral and dorsal walls of said first multiple-use portion, an inner surface of said second portion, configured to come into contact with the patient, being continuous.

4. A device for the treatment of patients by phototherapy according to claim 3, wherein the inner surface of the second single-use portion, configured to come into contact with a patient's back, is provided with a burying prevention system formed of a band intended to pass between patient's legs, and having a free end reversibly attachable to said patient.

5. A device for the treatment of patients by phototherapy according to claim 3, wherein free ends of one of the two pockets of the second single-use portion are provided with reversible attachment means capable of cooperating with the other pocket, and configured to limit leaks of the light emitted by said device towards the patient's head.

6. A device for the treatment of patients by phototherapy according to claim 1, wherein the first single-use portion is formed from a laminated bilayer membrane complying with the Oekotex 100 class-1 standard.

7. A device for the treatment of patients by phototherapy according to claim 6, wherein the inner layer of the multiple-use portion is a thin white breathable impermeable membrane configured to orient the light towards the patient and of preventing leaks to the outside of the device, and wherein the external layer of said multiple-use portion is dark-colored and blocks light likely to pass through the inner membrane towards the outside of said device.

8. A device for the treatment of patients by phototherapy according to claim 1, wherein the second single-use portion is made of a multi-material, the multi-material having an inner surface configured to come into contact with the patient, the multi-layer material configured to let the light emitted by said first multiple-use portion diffuse, the outer surface thereof which is in contact with said first multiple-use portion being impermeable.

9. A device for the treatment of patients by phototherapy according to claim 1, wherein the second single-use portion integrates a RFID-type emitter, configured to be activated by the light source to which the optical fibers are connected, and configured to control the presence of said single-use portion on the device and/or the occurrence of the prior use of said single-use portion for change of the single-use portion.

10. A device for the treatment of patients by phototherapy according to claim 1, wherein the light is formed of a LED, OLED diode or of a laser emitting blue light having a wavelength in the range from 420 to 500 nanometers.

11. A device for the treatment of patients by phototherapy according to claim 1, wherein a structure of the first multiple-use portion defines a light-emitting surface enabling to illuminate at least 60% of the surface area of the patient's body.
